(19) United States
(12) Patent Application Publication
(10) Pub. No.: US 2024/0325142 A1
(43) Pub. Date: Oct. 3, 2024

(54) Sealing Feature for Prosthetic Heart Valve

(71) Applicant: Cephea Valve Technologies, Inc., Abbott Park, IL (US)

(72) Inventors: Zachary R. Vidlund, Robbinsdale, MN (US); Jared Floersch, Minneapolis, MN (US)

(73) Assignee: Cephea Valve Technologies, Inc., Abbott Park, IL (US)

(21) Appl. No.: 18/398,831

(22) Filed: Dec. 28, 2023

Related U.S. Application Data
(60) Provisional application No. 63/493,410, filed on Mar. 31, 2023.

(51) Int. Cl. A61F 2/24

(52) U.S. Cl. CPC A61F 2/2418; A61F 2220/0025; A61F 2250/0028; A61F 2250/0036; A61F 2250/0039; A61F 2250/0076

(57) ABSTRACT

A prosthetic heart valve includes a sealing feature coupled to the inner frame at a coupling point and extending between the coupling point and a point of the outer frame that is radially outward of the coupling point in a direction substantially orthogonal to the longitudinal axis, the sealing feature comprising a plurality of layers.

### CROSS-REFERENCE TO RELATED APPLICATIONS

The present application claims the benefit of the filing date of U.S. Provisional Patent Application No. 63/493,410 filed Mar. 31, 2023, the disclosure of which is hereby incorporated herein by reference.

### BACKGROUND OF THE DISCLOSURE

Heart valve disease is a significant cause of morbidity and mortality. A primary treatment of this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after re-expansion.

Some prosthetic heart valves include a cuff, skirt, or another sealing member in the form of a woven fabric. It is generally important that sealing members, including woven fabric sealing members, do not fail during the operational life of the prosthetic heart valve.

### BRIEF SUMMARY

The present application overcomes the disadvantages of the prior art by providing a sealing feature with a plurality of layers with an angular offset. This allows the stiffer sections of the weave to be offset from each other when assembled, and more evenly distributes the loading across more points. This reduces the localized load and can remedy loading failures.

One aspect of the disclosure provides a prosthetic heart valve, comprising: an inner frame having a longitudinal axis; a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis; an outer frame connected to the inner frame; and a sealing feature coupled to the inner frame at a coupling point and extending between the coupling point and a point of the outer frame that is radially outward of the coupling point in a direction substantially orthogonal to the longitudinal axis, the sealing feature comprising a plurality of layers.

In one example, the sealing feature comprises a first layer and a second layer, the first layer and the second layer each being formed as woven layers.

In one example, there is an offset between a first weave pattern of the first layer and a second weave pattern of the second layer.

In one example, the offset comprises an angular offset between the first weave pattern and the second weave pattern.

In one example, the first layer defines a first notch and the second layer defines a second notch, such that alignment of the first notch and the second notch results in the angular offset.

In one example, the first weave pattern includes a first warp and first weft oriented at 90 degrees relative to each other, the second weave pattern includes a second warp and second weft oriented at 90 degrees relative to each other, and the angular offset is 45 degrees.

In one example, the first layer and the second layer are each substantially impermeable to blood flowing through the respective layer.

In one example, when the prosthetic heart valve is in an implanted condition during ventricular systole, areas of the first layer experiencing a maximum stress are aligned with areas of the second layer experiencing a minimum stress.

In one example, the sealing feature comprises an overlap region at which a first layer of the plurality of layers overlaps a second layer of the plurality of layers.

In one example, the sealing feature comprises a second region at which the first layer and the second layer do not overlap.

In one example, the second region is positioned radially inwardly relative to the overlap region.

In one example, the overlap region has a greater thickness than the second region.

In one example, a first layer of the plurality of layers and a second layer of the plurality of layers are coupled to the outer frame.

In one example, a first layer of the plurality of layers and a second layer of the plurality of layers are coupled directly to each other.

In one example, the first layer and the second layer are coupled directly to each other by a double running stitch pattern.

In one example, a second layer of the plurality of layers has an inner diameter that is greater than an inner diameter of a first layer of the plurality of layers.

In one example, the second layer is positioned nearer an inflow end of the prosthetic heart valve than is the first layer.

### BRIEF DESCRIPTION OF THE DRAWINGS

FIG. 1A is a side view of an assembled stent frame of a prosthetic heart valve of the prior art, the stent frame being shown in an expanded condition.

FIG. 1B is a side view of an outer frame of the stent frame of FIG. 1A.

FIG. 1C is a flattened view of the outer stent of FIG. 1B, as if cut longitudinally and laid out flat on a table in an unexpanded condition.

FIG. 1D is a side view of an inner frame of the stent frame of FIG. 1A.

FIG. 1E is a flattened view of the inner stent of FIG. 1D, as if cut longitudinally and laid out flat on a table in an unexpanded condition.

FIG. 2A is a side view of an assembled stent frame of a prosthetic heart valve according to an embodiment of the disclosure, the stent frame being shown in an expanded condition.

FIG. 2B is a side view of an outer frame of the stent frame of FIG. 2A.

FIG. 2C is a flattened view of the outer stent of FIG. 2B, as if cut longitudinally and laid out flat on a table in an unexpanded condition.

FIG. 2D is a side view of an inner frame of the stent frame of FIG. 2A.

FIG. 2E is a flattened view of the inner stent of FIG. 2D, as if cut longitudinally and laid out flat on a table in an unexpanded condition.

FIG. 3 is a cross-sectional schematic diagram of a prosthetic heart valve in the expanded condition and implanted relative to the atrium and ventricle.

FIG. 4A is a top view of a prosthetic heart valve incorporating a sealing feature according to one or more aspects of the disclosure.

FIG. 4B is a sectional side view along B-B of the prosthetic heart valve of FIG. 4A.

FIG. 4C is a sectional side view of a portion of the prosthetic heart valve of FIGS. 4A-B.

FIG. 5A is a schematic view of a plurality of layers of sealing feature.

FIG. 5B is a schematic view of first and second layers and sealing feature.

FIG. 5C is a schematic view of the first layer of the sealing feature of FIG. 5A, with annotations of regions of high stress and low stress.

FIG. 6 is a schematic cross-sectional diagram of the first layer and the second layer.

FIGS. 7 and 8 are perspective views of the sealing feature showing the coupling.

FIGS. 9A-B are schematic views of sealing features according to additional aspects of the disclosure.

FIG. 10 is a schematic view of an exemplary sealing feature according to one or more aspects of the disclosure.

FIG. 11 is a schematic view of an exemplary sealing feature according to one or more aspects of the disclosure.

### DETAILED DESCRIPTION

As used herein, the term “inflow end,” when used in connection with a prosthetic heart valve, refers to an end of the prosthetic heart valve into which blood first flows when the prosthetic heart valve is implanted in an intended position and orientation. On the other hand, the term “outflow end,” when used in connection with a prosthetic heart valve, refers to the end of the prosthetic heart valve through which blood exits when the prosthetic heart valve is implanted in an intended position and orientation. In the figures, like numbers refer to like or identical parts. As used herein, the terms “substantially,” “generally,” “approximately,” and “about” are intended to mean that slight deviations from absolute are included within the scope of the term so modified. When ranges of values are described herein, those ranges are intended to include sub-ranges. For example, a recited range of 1 to 10 includes 2, 5, 7, and other single values, as well as all sub-ranges within the range, such as 2 to 6, 3 to 9, 4 to 5, and others.

The present disclosure is generally directed to collapsible prosthetic mitral valves, and in particular various features of stents thereof to provide enhanced functionality. However, it should be understood that the features described herein may apply to other types of prosthetic heart valves, including prosthetic heart valves that are adapted for use in other heart valves, such as the tricuspid heart valve. Further, the features of the prosthetic heart valves described herein may, in some circumstances, be suitable for surgical (e.g., non-collapsible) prosthetic heart valves. However, as noted above, the disclosure is provided herein in the context of a collapsible and expandable prosthetic mitral valve.

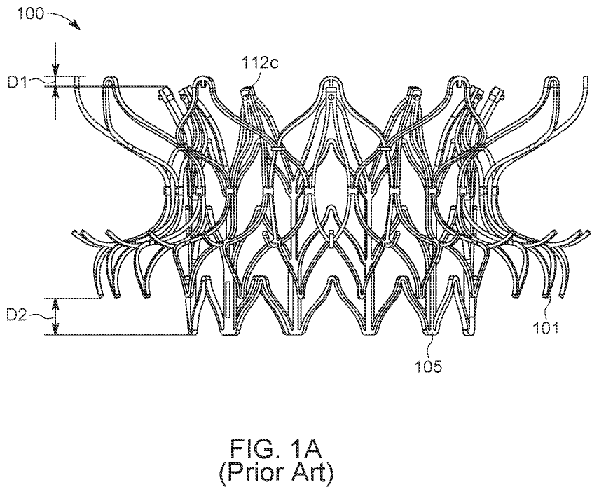

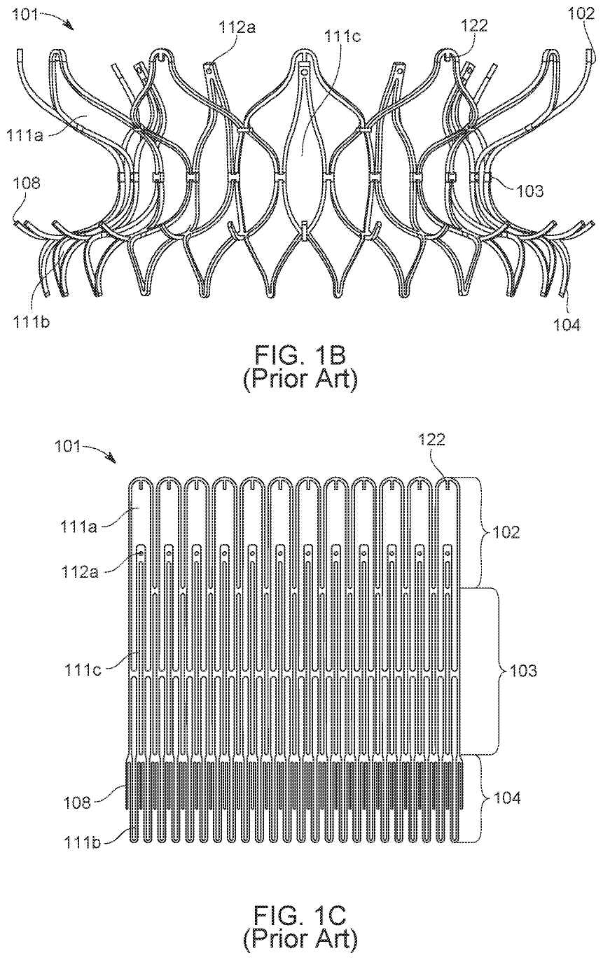

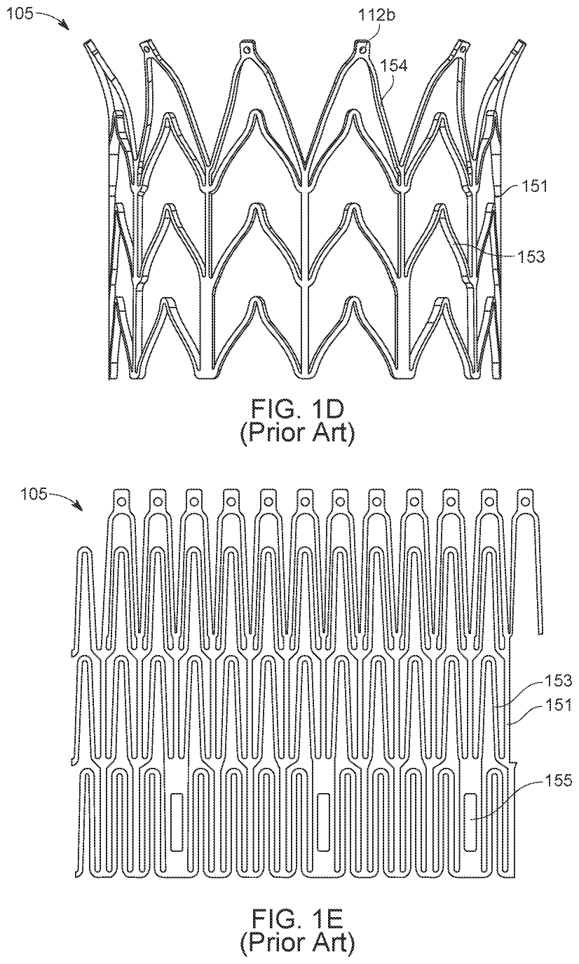

FIG. 1A illustrates an example of a collapsible and expandable prosthetic heart valve 100, according to the prior art, which may be particularly suited for replacement of a native mitral or tricuspid valve. It should be understood that the prosthetic heart valve 100 illustrated in FIG. 1A omits certain features that would typically be included, such as a valve assembly to assist in controlling blood flow through the prosthetic heart valve, and interior and/or exterior fabrics or tissue skirts to assist with providing a seal around the prosthetic heart valve and/or with enhancing tissue ingrowth to fix the prosthetic heart valve within the native heart valve over time. However, for purposes of simplicity, the prosthetic leaflet(s) and skirt(s) are omitted from the drawings for clarity of illustration.

The prosthetic heart valve 100 is illustrated in FIG. 1A in an expanded configuration. The stent of the prosthetic heart valve 100 may include an outer stent or frame 101 and an inner stent or frame 105 positioned radially within the outer frame. The outer frame 101 may be primarily for anchoring the prosthetic heart valve 100 within the native heart valve annulus, while the inner frame 105 may be primarily for holding the prosthetic valve assembly in the desired position and orientation.

Outer frame 101 is illustrated in FIGS. 1B-C isolated from other components of the prosthetic heart valve 100. In FIG. 1B, the outer frame 101 is illustrated in an expanded condition. In FIG. 1C, the outer frame 101 is illustrated in an unexpanded condition, as if cut longitudinally and laid flat on a table. As used herein, the term “unexpanded” refers to the state of the stent prior to being shape-set (e.g., immediately after it is cut from a tube of nitinol). After being formed, the stent may be shape-set to the expanded condition, and may also have a collapsed condition in which the stent is collapsed to a smaller size from its expanded condition. The shape of the stent may be similar, but not necessarily identical, in the unexpanded and collapsed conditions. As shown in FIGS. 1B-C, outer frame 101 may include an atrial portion or anchor 102, a ventricular portion or anchor 104, and a central portion 103 coupling the atrial portion to the ventricular portion. The central portion 103 may be between atrial portion 102 and ventricular portion 104. Atrial portion 102 may be configured and adapted to be disposed on an atrial side of a native valve annulus, and may flare radially outwardly from the central portion 103. Ventricular portion 104 may be configured and adapted to be disposed on a ventricle side of the native valve annulus, and may also flare radially outwardly from the central portion 103. The central portion 103 may be configured to be situated in the valve orifice, for example in contact with the native valve annulus. In use, the atrial portion 102 and ventricle portion 104 effectively overlie the native valve annulus on the atrial and ventricular sides thereof, respectively, helping to hold the prosthetic heart valve 100 in place.

The atrial portion 102 may be formed as a portion of a stent or other support structure that includes or is formed by a plurality of generally diamond-shaped cells, although other suitable cell shapes, such as triangular, quadrilateral, or polygonal may be appropriate. In some examples, the atrial portion 102 may be formed as a braided mesh, as a portion of a unitary stent, or a combination thereof. According to one example, the stent that includes the atrial portion 102 may be laser cut from a tube of nitinol and set to a desired shape, for example via heat treatment, so that the stent, including atrial portion 102, is collapsible for delivery, and re-expandable to the set-shape during deployment. The atrial portion 102 may be set, for example via heat treatment, to a suitable shape to conform to the native anatomy of the valve annulus to help provide a seal and/or anchoring between the atrial portion 102 and the native valve annulus. The shape-set atrial portion 102 may be partially or entirely covered by a cuff or skirt, on the luminal and/or abluminal surface of the atrial portion 102. The skirt may be formed of any suitable material, including biomaterials such as bovine pericardium, biocompatible polymers such as ultra-high molecular weight polyethylene, woven polyethylene terephthalate (“PET”) or expanded polytetrafluoroethylene (“ePTFE”), or combinations thereof. The atrial portion 102 may include features for connecting the atrial portion to a delivery system. For example, the atrial portion 102 may include pins or tabs 122 around which sutures (or suture loops) of the delivery system may wrap, so that while the suture loops are wrapped around the pins or tabs 122, the outer frame 101 maintains a connection to the delivery device.

The ventricular portion 104 may also be formed as a portion of a stent or other support structure that includes or is formed of a plurality of diamond-shaped cells, although other suitable cell shapes, such as triangular, quadrilateral, or polygonal may be appropriate. In some examples, the ventricular portion 104 may be formed as a braided mesh, as a portion of a unitary stent, or a combination thereof. According to one example, the stent that includes the ventricular portion 104 may be laser cut from a tube of nitinol and set to a desired shape, for example via heat treatment, so that the ventricular portion 104 is collapsible for delivery, and re-expandable to the set-shape during deployment. The ventricular portion 104 may be partially or entirely covered by a cuff or skirt, on the luminal and/or abluminal surface of the ventricular portion 104. The skirt may be formed of any suitable material described above in connection with the skirt of atrial portion 102. It should be understood that the atrial portion 102 and ventricular portion 104 may be formed as portions of a single support structure, such as a single stent or braided mesh. However, in other embodiments, the atrial portion 102 and ventricular portion 104 may be formed separately and coupled to one another.

As illustrated in FIG. 1A, the inner frame 105 may be positioned radially within the outer frame 101 when the inner and outer frames are assembled together. Inner frame 105 is illustrated in FIGS. 1D-E isolated from other components of the prosthetic heart valve 100. In FIG. 1D, the inner frame 105 is illustrated in an expanded condition. In FIG. 1E, the inner frame 105 is illustrated in an unexpanded condition, as if cut longitudinally and laid flat on a table. As shown in FIGS. 1D-E, the inner frame 105 may include a plurality of axially or longitudinally extending struts 151 and interconnecting v-shaped strut members 153. According to some embodiments, the inner frame 105 may have more or fewer v-shaped members 153 extending circumferentially around the diameter thereof than the number of cells in the atrial portion 102 and/or ventricular portion 104 of the outer frame 101, such as double or half the number. In some examples, the inner frame 105 may flare radially outwards at the atrial end, e.g., to conform to the flare of the atrial portion 102 of the outer frame 101. One or more prosthetic leaflets may be coupled to the inner frame 105 to form a prosthetic valve assembly, the prosthetic valve assembly configured to allow unidirectional flow of blood through the prosthetic valve assembly from the atrial end toward the ventricular end of the prosthetic heart valve 100. As best illustrated in FIG. 1E, the inner frame 105 may include a plurality of commissure windows 155 formed in axial struts 151. For example, inner frame 105 may include three generally rectangular-shaped commissure windows 155 equidistantly spaced around the circumference of the inner frame, with each commissure window adapted to provide a location for coupling two adjacent prosthetic leaflets to the axial strut 151. However, more or fewer commissure windows 155 may be provided depending on how many prosthetic leaflets will be coupled to the inner frame 105.

The outer frame 101 and/or the inner frame 105 may be formed of a superelastic and/or shape memory material such as nitinol. According to some examples, other biocompatible metals and metal alloys may be suitable. For example, superelastic and/or self-expanding metals other than nitinol may be suitable, while still other metals or metal alloys such as cobalt chromium or stainless steel may be suitable, particularly if the stent or support structure is intended to be balloon expandable. In some examples, the outer frame 101 and/or inner frame 105 may be laser cut from one or more tubes, such as a shape memory metal tube. The shape memory metal tube may be nitinol or any other bio-compatible metal tube. For example, the outer frame 101 may be laser cut from a first tube while the inner frame 105 may be laser cut from a second tube of smaller diameter.

The prosthetic heart valve 100 may be adapted to expand from a collapsed or constrained configuration to an expanded configuration. According to some examples, the prosthetic heart valve 100 may be adapted to self-expand, although the prosthetic heart valve could instead be partially or fully expandable by other mechanisms, such as by balloon expansion. The prosthetic heart valve 100 may be maintained in the collapsed configuration during delivery, for example via one or more overlying sheaths that restrict the valve from expanding. The prosthetic heart valve 100 may be expanded during deployment from the delivery device once the delivery device is positioned within or adjacent to the native valve annulus. In the expanded configuration, the atrial portion 102 and ventricular portion 104 may extend radially outward from a central longitudinal axis of the prosthetic heart valve 100 and/or central portion 103, and may be considered to flare outward relative to the central longitudinal axis of the replacement valve and/or central portion 103. The atrial portion 102 and ventricular portion 104 may be considered flanged relative to central portion 103. The flared configuration of atrial and ventricular portions 102, 104 relative to central portion 103 is described in the context of a side view of the outer frame 101, as can be best seen in FIG. 1B. In some embodiments, the flared configuration of the atrial and ventricular portions 102, 104 and the central portion 103 may define a general hour-glass shape in a side view of the outer frame 101. That is, the atrial and ventricular portions 102, 104 may be flared outwards relative to the central portion 103 and then curved or bent to point at least partially back in the axial direction. It should be understood, however, that an hour-glass configuration is not limited to a symmetrical configuration.

The outer frame 101 may be configured to expand circumferentially (and radially) and foreshorten axially as the prosthetic heart valve 100 expands from the collapsed delivery configuration to the expanded deployed configuration. As described herein, the outer frame 101 may define a plurality of atrial cells 111a in one circumferential row and a plurality of ventricular cells 111b in another circumferential row. Each of the plurality of cells 111a, 111b may be configured to expand circumferentially and foreshorten axially upon expansion of the outer frame 101. As shown, the cells 111a-b may each be diamond-shaped. In the illustrated embodiment, the outer frame 101 includes twelve atrial cells 111a, and twenty-four ventricular cells 111b. In addition, a third plurality of cells 111c may be provided in another circumferential row. Cells 111c may have a first end that is within a corresponding atrial cell 111a, at least when the frame is collapsed (similar to the unexpanded condition shown in FIG. 1C). Cells 111c may have a second end that is positioned between pairs of adjacent ventricular cells 111b, at least when the frame is collapsed (similar to the unexpanded condition shown in FIG. 1C). In this particular example, the outer frame 101 includes twelve center cells 111c.

Still referring to FIGS. 1B-C, a pin or tab 122 may extend from an apex of each atrial cell 111a in a direction toward the outflow end of the outer frame 101. Although one pin or tab 122 is illustrated in each atrial cell 111a, in other embodiments fewer than all of the atrial cells may include a pin or tab. Each center cell 111c may include an aperture 112a or other coupling feature at a first apical end thereof for coupling to the inner frame 105, as is described in greater detail below. In the illustrated embodiment, the aperture 112a is positioned at the inflow apex of center cells 111c, and each center cell includes an aperture, although in other embodiments fewer than all of the cells may include such apertures. In the expanded condition of the outer frame 101, as shown in FIG. 1B, the apex of the center cells 111c that include the apertures 112a may be positioned radially inwardly of the apex of the atrial cells 111a near the inflow end of the outer frame. In addition, each center cell 111c may include a tine or barb 108 extending from the opposite apex on the outflow end of the center cell, although fewer than all of the center cells may include such barbs. In the collapsed condition of the outer frame 101 (similar to the unexpanded condition shown in FIG. 1C), each barb 108 extends toward the outflow end of the outer frame, each barb being positioned between two adjacent ventricular cells 111b. In the expanded condition of the outer frame 101, as shown in FIG. 1B, the barbs 108 may hook upwardly back toward the inflow end, the barbs being configured to pierce native tissue of the valve annulus, such as the native leaflets, to help keep the prosthetic heart valve from migrating under pressure during beating of the heart. Typically, the term “tine” may refer to a structure configured to pierce into (or otherwise frictionally engage) tissue, while the term “barb” may refer to a tine that also includes a barb-like structure to prevent the barb from pulling out of the tissue once pierced. However, as used herein, the term “barb” includes tines, with or without actual “barb”-like structures that prevent pulling out of tissue, unless specifically noted otherwise.

The inner frame 105 may be configured to expand circumferentially (and radially) while maintaining the same (or about the same) axial dimension (e.g., be non-foreshortening) as the prosthetic heart valve 100 expands from the collapsed delivery configuration to the expanded configuration. The axial struts 151 may contribute to this non-foreshortening functionality. By being non-foreshortening, the inner frame 105 may prevent (or reduce) strain from being placed on the prosthetic leaflets when the inner frame 105 transitions between the collapsed and expanded conditions. Thus, while the outer frame 101 may be designed to be foreshortening, the inner frame 105 may be designed so as to be substantially non-foreshortening.

Inner frame 105 may include twelve longitudinal struts 151, with three rows of twelve v-shaped members 153. However, in other embodiments, more or fewer longitudinal struts 151 may be included, and more or fewer rows of v-shaped members 153 may be included. In the illustrated embodiment, the number of longitudinal struts 151 is equal to the number of atrial cells 111a of the outer frame 101. In addition, v-shaped coupling members 154 may extend from each adjacent pair of longitudinal struts 151. These v-shaped coupling members 154 may have half-diamond shapes, with the apex of each half-diamond shape including an aperture 112b, the v-shaped coupling members generally flaring radially outwardly in the expanded condition of inner frame 105.

Referring back to FIG. 1A, in the expanded conditions of the outer frame 101 and the inner frame 105, the top portion of the center cells 111c may flare outwardly with a contour that substantially matches the outward flare of the v-shaped coupling members 154, so that apertures 112a and 112b align with each other. A coupling member, such as a rivet 112c, may pass through apertures 112a and 112b to couple the outer frame 101 to the inner frame 105.

Additional features and example replacement valves may be described in International patent application publication WO/2018/136959, filed Jan. 23, 2018, and titled “REPLACEMENT MITRAL VALVES,” which is hereby incorporated by reference herein.

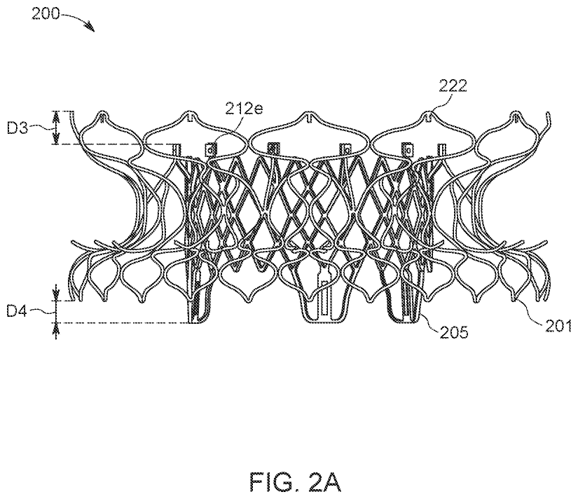

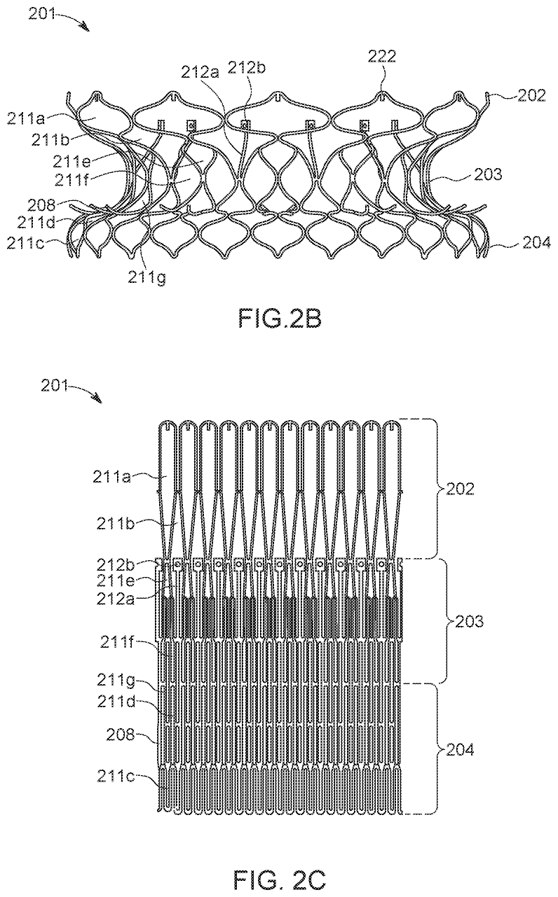

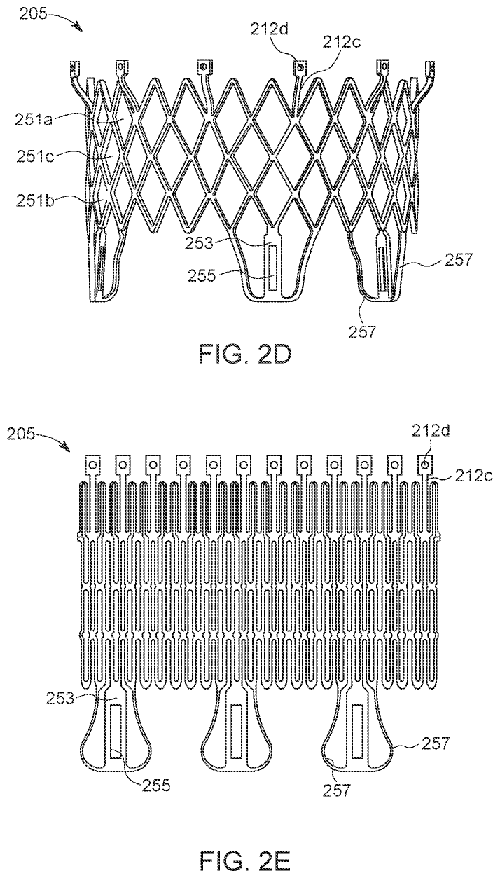

FIG. 2A illustrates another embodiment of a collapsible and expandable prosthetic heart valve 200, which may be particularly suited for replacement of a native mitral or tricuspid valve. The overall general structure of prosthetic heart valve 200 may be substantially similar to that of prosthetic heart valve 100 in both structure and function, but prosthetic heart valve 200 may have various differences to provide for certain benefits compared to prosthetic heart valve 100. For the purpose of brevity, only the differences between prosthetic heart valve 200 compared to prosthetic heart valve 100 are described in detail below, with the remaining features of prosthetic heart valve 200 being similar or identical to the corresponding features of prosthetic heart valve 100. As with prosthetic heart valve 100, it should be understood that the prosthetic heart valve 200 illustrated in FIG. 2A omits certain features such as prosthetic leaflets and luminal and/or abluminal stent skirts. The prosthetic heart valve 200 is illustrated in FIG. 2A in an expanded configuration. The stent of the prosthetic heart valve 200 may include an outer stent or frame 201 and an inner stent or frame 205 positioned radially within the outer frame.

Outer frame 201 is illustrated in FIGS. 2B-C isolated from other components of the prosthetic heart valve 200. In FIG. 2B, the outer frame 201 is illustrated in an expanded condition. In FIG. 2C, the outer frame 201 is illustrated in an unexpanded condition, as if cut longitudinally and laid flat on a table. Similar to outer frame 101, outer frame 201 may include an atrial portion or anchor 202, a ventricular portion or anchor 204, and a central portion 203 coupling the atrial portion to the ventricular portion.

The outer frame 201 may be configured to expand circumferentially (and radially) and foreshorten axially as the prosthetic heart valve 200 expands from the collapsed delivery configuration to the expanded deployed configuration. The outer frame 201 may define a plurality of atrial cells 211a, 211b in two circumferential rows. For example, the first row of atrial cells 211a may be generally diamond shaped and positioned on the inflow end of the outer frame 201. The second row of atrial cells 211b may be positioned at least partially between adjacent atrial cells 211a in the first row, with the atrial cells 211b in the second row being positioned farther from the inflow end than the first row of atrial cells 211a. The outer stent 201 may include twelve atrial cells 211a in the first row each having a diamond-shape, and twelve atrial cells 211b in the second row each having a skewed diamond shape. This skewed diamond shape, which is wider nearer the inflow (or top) end and narrower nearer the outflow (or bottom) end, may assist in transitioning from twelve cells per row on the atrial side of the stent to twenty-four cells per row on the ventricular side.

The outer frame 201 may include a plurality of ventricular cells 111c in a first row, and another plurality of ventricular cells 11d in a second row. The first row of ventricular cells 211c may be at the outflow end of the outer frame 201, and the second row of ventricular cells 211d may be positioned farther from the outflow end than, and adjacent to, the first row of ventricular cells 211c. In the illustrated embodiment the first and second rows of ventricular cells 211c, 211d are all generally diamond-shaped and have substantially the same, or an identical, size, with twenty-four cells in the first row of ventricular cells 211c and twenty-four cells in the second row of ventricular cells 211d.

Outer stent 201 is also illustrated as including three rows of center cells. A first row of center cells 211e is positioned adjacent the atrial end of the outer stent 201, each cell 211e being positioned between a pair of adjacent atrial cells 211b. Each center cell 211e may be substantially diamond-shaped, but it should be understood that adjacent center cells 211e do not directly touch one another. The first row of center cells 211e may include twelve center cells 211e, with the combination of atrial cells 211b and the center cells 211e helping transition from rows of twelve cells on the atrial side to rows of twenty-four cells on the ventricular side. A second row of center cells 211f may be positioned at a longitudinal center of the outer frame 201, each center cell 211f being positioned between an atrial cell 211b and center cell 211e. In the illustrated embodiment, center cells 211f in the second row may be diamond-shaped, with the second row including twenty-four center cells 211f. Finally, a third row of center cells 211g may be positioned between the second row of center cells 211f and the second row of ventricular cells 211d. The third row of center cells 211g may include twenty-four cells and they may each be substantially diamond-shaped.

All of the cells 211a-g may be configured to expand circumferentially and foreshorten axially upon expansion of the outer frame 201. Similar to outer frame 100, a pin or tab 222 may extend from an apex of each atrial cell 211a in the first row in a direction toward the outflow end of the outer frame 201. Although one pin or tab 222 is illustrated in each atrial cell 211a in the first row, in other embodiments fewer than all of the atrial cells in the first row may include a pin or tab. Whereas outer frame 101 included apertures 112a at an apex of a center cell 111c, outer frame 201 may instead include coupling arms 212a. Each coupling arm 212a may be a strut that is coupled to a bottom or outflow apex of each atrial cell 211b in the second row, with each strut extending toward the inflow end of the outer frame 201 to a free end of the coupling arm 212a. The free end of each coupling arm 212a may include an aperture 212b for coupling to the inner frame 205, as described in greater detail below. In the collapsed condition (similar to the unexpanded condition shown in FIG. 2C), each coupling arm 212a is substantially surrounded by an atrial cell 211b in the second row. In the expanded condition, best shown in FIG. 2B, the coupling arms 212a may extend radially inwardly and have a contour so that the free end extends substantially parallel to the center longitudinal axis of the outer frame 201. In addition, outer frame 201 may include a plurality of tines or barbs 208 extending from a center portion or ventricular portion of the outer frame for piercing (or otherwise frictionally engaging) native tissue in the native annulus or in the native leaflets. In the illustrated embodiment, each barb 208 is connected to a ventricular cell 211d in the second row. In some embodiments, the barb 208 may be coupled to an inflow or outflow apex of each cell. In the particular illustrated embodiment, the barbs 208 are couple to ventricular cells 211d on an inflow half of the cell, on either side of the inflow apex. For example, the barb 208 in one ventricular cell 211d may be coupled to the inflow half of that cell on a right side of the apex, with the adjacent ventricular cell 211d having a barb coupled to the inflow half of that cell on a left side of the apex. With this configuration, the barbs 208 are provided in pairs with relatively little space between the barbs of a pair, but a relatively large space between adjacent pairs. However, it should be understood that the barbs 208 may in other embodiments be centered with even spacing between adjacent barbs, similar to that shown and described in connection with FIG. 1B. In the collapsed condition of the outer frame 201 (similar to the unexpanded condition shown in FIG. 2C), each barb 208 extends toward the outflow end of the outer frame, each barb being positioned within a ventricular cell 211d in the second row. In the expanded condition of the outer frame 201, as shown in FIG. 2B, the barbs 208 may hook upwardly back toward the inflow end, the barbs being configured to pierce native tissue of the valve annulus, such as the native leaflets, to help keep the prosthetic heart valve from migrating under pressure during beating of the heart.

As illustrated in FIG. 2A, the inner frame 205 may be positioned radially within the outer frame 201 when the inner and outer frames are assembled together. Inner frame 205 is illustrated in FIGS. 2D-E isolated from other components of the prosthetic heart valve 200. In FIG. 2D, the inner frame 205 is illustrated in an expanded condition. In FIG. 2E, the inner frame 205 is illustrated in an unexpanded condition, as if cut longitudinally and laid flat on a table. Whereas inner frame 105 includes longitudinal struts 151 and is non-foreshortening, inner frame 205 instead includes a plurality of rows of diamond-shaped cells so that the inner frame 205 foreshortens upon expansion. In the illustrated example, inner frame 205 includes three rows of diamond-shaped cells, including a first row of cells 251a at the inflow end of the inner frame, a second row of cells 251b at the outflow end of the inner frame, and a third row of cells 251c positioned between the first and second rows. In some embodiments, the inner frame 205 may include more or fewer rows of cells. In the expanded condition shown in FIG. 2D, the three rows of cells 251a-c may be substantially cylindrical.

One or more prosthetic leaflets may be coupled to the inner frame 205 to form a prosthetic valve assembly, the prosthetic valve assembly configured to allow unidirectional flow of blood through the prosthetic valve assembly from the atrial end toward the ventricular end of the prosthetic heart valve 200. As illustrated in FIGS. 2D-E, the inner frame 205 may include a plurality of commissure windows 255 formed in axial struts 253 extending from selected cells 251b at the outflow end of the inner frame 205. For example, inner frame 205 may include three generally rectangular shaped commissure windows 255 equidistantly spaced around the circumference of the inner frame, with each commissure window adapted to provide a location for coupling two adjacent prosthetic leaflets to the axial strut 253. However, more or fewer commissure windows 255 may be provided depending on how many prosthetic leaflets will be coupled to the inner frame 205. Additional support struts 257 may connect the axial struts 253 to the cells 251b. In particular, a first support strut 257 may couple the outflow end of each axial strut 253 to the outflow apex of a first cell 251b on a first side of the axial strut, and a second support strut 257 may couple the outflow end of each axial strut 253 to the outflow apex of a second cell 251b on a second opposite side of the axial strut, with the axial strut coupled to a third cell 251b between the first and second cells. As shown in FIGS. 2D-E, the support struts 257 may be contoured so as to avoid presenting any sharp tips, which may help avoid damaging the anatomy.

The inner frame 205 may also include a plurality of coupling arms 212c. Each coupling arm 212c may have a first end coupled to the inner frame 205 at an inflow end of the inner frame. In particular, the first end of each coupling arm 212c may be attached to a junction between two adjacent cells 251a in the first row at the inflow end. The coupling arms 212c may extend in a direction away from the outflow end of the inner frame 205 to a free end, with the free end including an aperture 212d therein. In the expanded condition, as shown in FIG. 2D, the coupling arms 212c may initially extend radially outwardly from the inner frame 205, with the free end being contoured so that the free end extends substantially parallel to the longitudinal axis of the inner frame 205. In the illustrated embodiment, inner frame 205 may include a total of twelve coupling arms 212c spaced equidistantly around the circumference of the inner frame. Preferably, the number of coupling arms 212c corresponds to the number of coupling arms 212a. Referring back to FIG. 2A, a coupling member, such as a suture or a rivet 212e, may pass through apertures 212b and 212d to couple the outer frame 201 to the inner frame 205.

Various differences between prosthetic heart valves 100 and 200 are now described in greater detail. Outer frame 101 may include a relatively small number of cells which each define a relatively large area. For example, outer frame 101 includes only two rows of cells 11a, 111b for anchoring (which excludes the row of cells 111c which in large part serve to connect the outer frame 101 to the inner frame 105). On the other hand, despite having a generally similar profile as outer frame 101, outer frame 201 includes a larger number of cells that typically define a smaller area. For example, outer frame 201 may be thought of as including six rows of full cells (if cells 211b and 211e are counted as a single row considering the circumferential overlap between those cells). One result of this difference is that the outer frame 101 may have relatively little redundancy compared to outer frame 201. Thus, in the event that a strut defining a cell (or a portion thereof) fractures, the likelihood of stent failure (or the likely detrimental effect of a failure) may be significantly reduced in outer stent 201 compared to outer stent 101, due to increased redundancy in the design. Another benefit of the increased number of cells in outer stent 201 compared to outer stent 101 concerns any tissue and/or fabric skirts coupled to the outer stent 201. For example, due to additional stent structure, more options may be available for how and where to attach tissue and/or fabric skirts to the outer stent 201. This additional stent structure may also better distribute the pressure applied by outer stent 201 to the patient's tissue, thereby reducing the risk of tissue erosion. Still further, the greater number of cells, and smaller area of cells, of outer stent 201 compared to outer stent 101, may allow for reduced forces required to collapse the outer stent 201 during loading into a delivery device, and also reduce forces experienced during deploying the outer stent. This may result in lower strain experienced by the outer stent 201, compared to outer stent 101, and thus improve durability of the outer stent 201. Stated in another way, having more cells with a desired aspect ratio may allow for each cell to have a relatively small strut width, while still being able to maintain a desired stiffness. The diamond cell pattern may allow for the cells (and the stent) to collapse without significant twisting or torsion. This type of twisting or torsion, which may be a primary driver of higher strain, may be more likely to occur in outer stent 101 compared to outer stent 201. The smaller strut width and reduced twisting may provide lower strains, allows sheathing to smaller diameters and improvements in durability

There are various additional differences between prosthetic heart valves 100 and 200, including between the inner stents 105 and 205. For example, inner stent 105 includes commissure windows 155 in axial struts 151 that form part of the outflow end of the inner stent 105, whereas the commissure windows 255 of inner stent 205 extend beyond the main body of the remainder of the inner stent 205. This may allow the main body of inner stent 205 to be shorter than the main body of inner stent 105. In turn, the inner frame 205 may be able to extend a distance D4 beyond the outflow end of the outer stent 201 (see FIG. 2A) that is smaller than the distance D2 that the inner frame 105 extends beyond the outflow end of the outer stent 101 (see FIG. 1A). This may be desirable because, if there is less structure extending into the left (or right) ventricle, there is a smaller likelihood that the inner stent 205 will obstruct the left (or right) ventricular outflow tract, compared to inner stent 105. In other words, there is little or no structure between adjacent commissure windows 255 that would obstruct blood flow, while there is a relatively larger amount of stent structure between adjacent commissure windows 155. Additionally, during ventricular systole, there is relatively small amount of stent structure blocking blood from pressing against the outflow end of the prosthetic leaflets, meaning that the prosthetic leaflets may be faster to coapt with each other during ventricular systole as a result of the extension of the commissure windows 255 farther than adjacent stent structure. The shorter distance of the main body of the inner stent 205 may also provide for greater maneuverability of prosthetic heart valve 200 compared to prosthetic heart valve 100 during deployment and/or repositioning of the prosthetic heart valve. However, the design of inner stent 205 may result in the commissure windows 255 being more likely to deflect during use compared to commissure windows 155 of inner stent 105. In other words, commissure windows 255 may be more cantilevered than commissure windows 155. Thus, when the prosthetic valve assembly is under pressure, particularly when the prosthetic leaflets are closed and are resisting retrograde blood flow, the commissure windows 255 may have a greater tendency to deflect inwardly compared to commissure windows 155. To mitigate this possibility, the commissure windows 255 may include additional supports, in the form of support struts 257 described above, to form a “webbed commissure” structure. This “webbed commissure” structure may also provide a relatively atraumatic structure at the commissure windows 255, which may help avoid piercing any native tissue. Thus, the design of inner stent 205, including that of the commissure windows 255 and the support struts 257, allows for a relatively small protrusion of the inner stent 205 into the ventricle while also helping achieve optimal deflection of the commissure windows 255. These webbed commissures may also provide additional benefits to leaflet closure due to better fluid access to the free margin of the leaflets and a more robust sewing pattern at the commissures such that a metal retaining plate is not required and durability is improved.

Another difference between inner frame 105 and 205 is the position and structure of the v-shaped coupling members 154 (which include coupling aperture 112b) compared to coupling arms 212c (which include coupling aperture 212d). For example, v-shaped coupling members 154 are coupled to the main body of the inner frame 105 at two locations (the two struts that form the “v”-shape), whereas coupling arms 212c are coupled to the main body of inner frame 205 at only one location. This coupling at one location may be robust from a durability standpoint, while also avoiding twisting of struts during expansion and/or shape-setting. For relatively large sized prosthetic heart valves (which include relatively large frames), this may be especially important because the distance between the inner and outer frames may be relatively large. Further, coupling arms 212c may be coupled to inner frame 205 so that aperture 212d extends beyond the inflow end of the inner frame 205a distance (see FIG. 2A) that is smaller than the distance which aperture 112b extends beyond the inflow end of the inner frame 105 (see FIG. 1A). At least partially as a result of this, the inflow end of the outer frame 201 extends a distance D3 beyond the location of the coupling rivets 212e (see FIG. 2A) that is larger than the distance D1 which the inflow end of the outer frame 101 extends beyond the location of the coupling rivets 112c (see FIG. 1A). Release from the delivery system may be highly dependent on the angle of the pin or tab 222 relative to the axis and the distance D3. A central lumen that connects all the suture loops is pushed downward to release the suture loops. The angle from the pin or tab 222 to the interfering inner frame 205 may be important for release. The larger the angle, the easier the release. Thus, as can be best seen by comparing FIG. 2A to FIG. 1A, there is a relatively large amount of clearance in the atrial cells 211a around pin or tab 222 compared to the amount of clearance in the atrial cells 111a around pin or tab 122. As noted above, during deployment of the prosthetic heart valve 200, sutures or suture loops may loop around pins or tabs 222 to maintain a physical connection between the prosthetic heart valve and the delivery device. After deployment of the prosthetic heart valve 200, the suture loops may be advanced to slip the suture loops off the pins or tabs 222 to fully disconnect the prosthetic heart valve 200 from the delivery device. The larger amount of clearance around the pins or tabs 222, compared to the amount of clearance around pins or tabs 122, may make this process easier and reduce the likelihood of the sutures or suture loops failing to disconnect from the pins or tabs 222, for example via obstruction with other nearby stent structure.

For each of the frames 101, 105, 201, 205 described above, the wall thickness of each individual stent may be substantially constant, whether or not the inner frames 105, 205, have the same wall thicknesses as the corresponding outer frames 101, 201. However, in some embodiments, the stents that form the prosthetic heart valve 200 may have varying wall thickness. For example, the wall thickness of the outer stent 201 near the inflow end and/or near the outflow end may be reduced relative to the wall thickness of the remainder of the outer stent to reduce the stiffness of the atrial and/or ventricular tips of the outer stent 201 to reduce the likelihood of causing trauma to the tissue. For example, the wall thickness of the outer stent in the atrial cells 211a of the first row may be reduced compared to the remainder of the outer stent 201. In one example, only about half the atrial cells 211a in the first row, for example the inflow half, may have a reduced wall thickness compared to the remainder of the outer stent 201. The decrease in stent wall thickness may be gradual or abrupt, for example via a step change. The decreased thickness areas of the outer stent 201 may be created by any suitable method, including for example forming the outer stent 201 of a constant thickness, and then grinding down the stent to a smaller thickness at the desired locations. Additionally, or alternatively, the ventricular cells 211c in the first row may have a reduced wall thickness compared to the remainder of the outer stent 201. As with atrial cells 211a, the wall thickness of ventricular cells 211c may be reduced either gradually or abruptly, including at about half the length of the ventricular cells 211c on the outflow portion of those ventricular cells 211c. With this configuration, one or both tip ends of the outer stent 201 may be provided with reduced thickness to provide reduced stiffness relative to other portions of the outer stent 201 in order to reduce trauma to native tissue. It should further be noted that the atrial and ventricular tip ends of the outer stent 201 may be generally low strain locations compared to the other portions of the outer stent 201. As a result, reducing the stent wall thickness at these locations may not substantially hinder durability of the stent.

In addition or alternatively to reducing the stent wall thickness at one or both tip ends of the outer stent 201, the stent wall thickness near the central waist portion of the outer stent 201 may be increased relative to other portions of the outer stent 201 in order to increase stiffness in this area. For example, the second row of center cells 211f may have a stent wall thickness that is larger than immediately adjacent areas of the stent body 201. For example a portion of the center cells 211f, or the entire center cells 211f, which may include portions of adjacent cells 211b, 211e, 211g, may have a wall thickness greater than all remaining portions of the outer stent 201. When prosthetic heart valve 200 is implanted into a native valve annulus, as noted above, this center portion may be in contact with the native valve annulus while the atrial and ventricular ends wrap around the native valve annulus. As a result, when the heart contracts to pump blood, the center waist portion of the outer stent 201 may be subjected to a relatively large amount of contractile forces. While it may be desirable for the outer stent 201 to have some level of flexibility so as conform to the shape of the native valve annulus, it is typically not desirable for forces imparted on the outer stent 201 to transfer to the inner stent 205 and affect the prosthetic valve leaflets therein. Thus, by increasing stent wall thickness in the waist area of the outer stent 201, deformation of the outer stent 201 may be reduced during beating of the heart, which may help reduce any resulting deformation of the inner stent 205 and prosthetic leaflets positioned therein. The stent wall thickness of the waist portion of the outer stent 201 may be increased by any desired method. For example, the entire outer stent 201 may be formed with a constant thickness that is equal to the desired thickness of the waist portion, and the remaining areas of the outer stent 201 may be ground down to a smaller stent wall thickness. In other embodiments, the waist portion of outer stent 201 may be subsequently increased after the outer stent is formed having a constant stent wall thickness, for example by additive manufacturing, spray coating, dip coating, or any other suitable modalities.

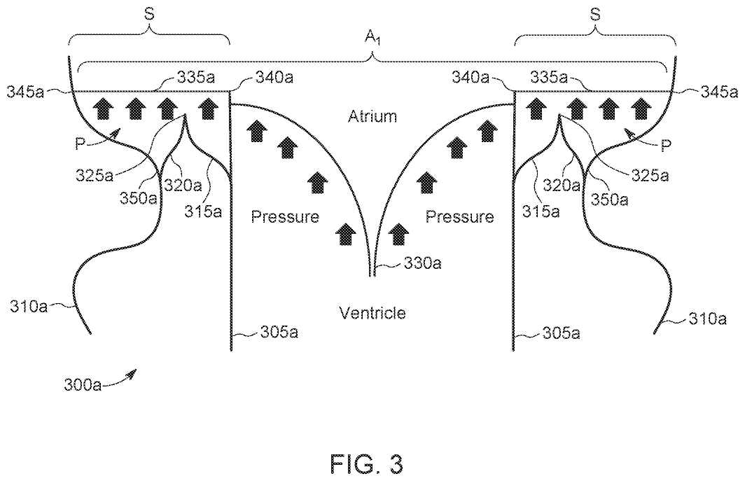

FIG. 3 is a cross-sectional schematic diagram of a prosthetic heart valve 300a in the expanded condition and implanted relative to the atrium and ventricle.

As shown, the prosthetic heart valve 300a can include an inner frame 305a (e.g., inner frame 105 or inner frame 205 described above) positioned radially within an outer frame 310a (e.g., outer frame 101 or outer frame 201). The inner frame 305a can be coupled with the outer frame 310a by any type of structural engagement. In one example, the inner frame 305a includes at least one coupling arm 315a that can be coupled to at least one respective coupling arm 320a of the outer frame 310a at an anchoring point 325a such that the inner frame 305a is anchored to the outer frame 310a on an atrial side of the outer frame 310a. In some examples, coupling arms 315a may be generally similar or identical to coupling arms 212c, and coupling arms 320a may be generally similar or identical to coupling arms 212a. However, other specific implementations of coupling arms 315a, 320a may be suitable for use with prosthetic heart valve 300a. For example, other exemplary coupling configurations are described in U.S. patent application Ser. No. 17/712,290 to Bergin, filed Apr. 4, 2022, entitled Frame Features for Transcatheter Mitral Valve Replacement Device, the entirety of which is hereby incorporated by reference. As another example, the coupling arms 315a, 320a can each be a double-arm connection (as opposed to a single arm configuration depicted in FIGS. 2A-B) as described in U.S. Patent Application Ser. No. 63/484,017 to Vidlund, filed Feb. 9, 2023, entitled Prosthetic Heart Valve Frame with Double-Arm Connection, the entirety of which is hereby incorporated by reference.

The prosthetic heart valve 300a may also include a plurality of prosthetic leaflets 330a (e.g., tissue leaflets) coupled to the inner frame 305a. The plurality of leaflets 330a are movable between an open configuration (not shown) and a closed configuration (as shown in FIG. 3) in which the leaflets 330a coapt, or meet in sealing abutment. In some embodiments, the prosthetic heart valve 300a includes three prosthetic leaflets 330a, with each pair of adjacent leaflets being coupled to each other and to the inner frame 305a at a commissure feature, such as a feature like commissure window 155 or 255.

For the leaflets 330a to allow unidirectional flow of blood through the prosthetic valve heart valve 300a from the atrial end toward the ventricular end of the prosthetic heart valve 300a, a two-dimensional annular span (represented as S in FIG. 3) between the inner frame 305a and the outer frame 310a is covered with a sealing feature 335a (also referred to as a sealing member). In this regard, the sealing feature 335a can extend or span an entire radial distance, in a direction substantially orthogonal to the longitudinal axis of the prosthetic heart valve 300a, between coupling point 340a of inner frame 305a and coupling point 345a of outer frame 310a. The sealing feature 335a can be in the shape of a two-dimensional annulus, thereby defining an inner perimeter near the inner frame 305a and an outer perimeter near the outer frame 310a.

The sealing feature 335a can couple with both the inner frame 305a and the outer frame 310a. The sealing feature 335a may couple with the inner frame 305a at any desired portion of the inner frame 305a, and in one example couples at a coupling point 340a that is the atrial-most point of the inner frame 305a. In this regard, the coupling point 340a is positioned radially inward, and on an atrial side, of an anchoring point 325a at which inner frame 305a couples with outer frame 310a. In another example, the coupling point 340a is any point on the inner frame 305a that is on an atrial side of anchoring point 325a, thereby providing the sealing feature 335a clearance with respect to the anchoring point 325a.

The sealing feature 335a may couple with the outer frame 310a at any desired portion of the outer frame 310a, and in one example couples at a coupling point 345a that is between the atrial-most point of the outer frame 310a and a point 350a from which coupling arm 325a extends inward from outer frame 310a. In this regard, the coupling point 345a may be positioned radially outward with respect to coupling point 340a. Further, the coupling point 345a may be positioned both radially outward, and on an atrial side, of an anchoring point 325a at which inner frame 305a couples with outer frame 310a. In the illustrated embodiment, the sealing feature 335a extends in a direction that is generally orthogonal to the direction of blood flow, but in other embodiments, the sealing feature 335 may extend at an oblique angle relative to the direction of blood flow.

The sealing feature 335a can extend and cover the entire two-dimensional annular span S between the coupling point 340a at inner frame 305a and the coupling point 345a at outer frame 310a, thereby defining a partially bounded three-dimensional annular pocket (represented as P in FIG. 3). The partially bounded three-dimensional annular pocket P is bounded in part by inner frame 305a (including any cuffs or skirts on the inner frame), outer frame 310a (including any cuffs or skirts on the outer frame), and sealing feature 335a. As depicted in FIG. 3, the partially bounded three-dimensional annular pocket P may also be partially bounded by coupling arm 315a and coupling arm 320a at certain locations, as the coupling arm 315 and coupling arm 320a are arranged in a spaced apart manner around a circumference of the prosthetic heart valve 300a. The pocket P is partially bounded insofar as the pocket P is relatively open on a ventricular or outflow side between the inner frame 305a and the portion of outer frame 310a of least diameter (which in the illustrated embodiment is a central waist of the outer frame 310a).

The sealing feature 335a can be formed at least in part, or in its entirety, of any suitable material that is substantially impermeable to blood and thereby prevents blood flow across the prosthetic heart valve 300a and through the sealing feature 335a when the prosthetic leaflets 330a are in the closed or coapted condition. The sealing feature 335a can be formed of a tissue or a fabric (such as a woven fabric), including synthetic fabrics, such as PET, PTFE, etc.

In a typical tricuspid or mitral valve, the configuration of sealing feature 335a results in a large area for backpressure, resulting from ventricular systole, to act against. The relevant area against which pressure acts is shown as cross-sectional area, represented as A₁, in the cross-section of FIG. 3. This cross-sectional area A₁ is the combination of the cross-sectional area of the leaflets 330a (i.e., area of 2D projection of leaflets onto a plane that is orthogonal to direction of blood flow) in addition to the cross-sectional area of the sealing feature 335a. In other words, because the leaflets 330a and the sealing feature 335a are both substantially impermeable to blood flow, as the leaflets 330a coapt during ventricular systole and the pressure within the ventricle is significantly higher than the pressure within the atrium, both the leaflets 330a and the sealing feature 335a are the surfaces that separate the high pressure of the ventricle from the low pressure of the atrium. In order for the prosthetic heart valve 300a to remain in a stable position within the native valve annulus, the leaflets 330a and sealing feature 335a must resist this pressure gradient. The construction of the outer frame 310a typically results in a very strong fixation of the outer frame 310a to the native valve annulus. However, the inner frame 305a is only coupled to the outer frame 310a via coupling arms 315a, 320a and sealing feature 335a. Thus, the backpressure during ventricular systole may have the most significant effect on the inner frame 305a, tending to move the inner frame 305a slightly in the atrial direction during ventricular systole. The resulting force on the inner frame 305a during ventricular systole is proportional, or equal, to the backpressure multiplied by the area A₁ against which the pressure acts. As noted above, this resulting force can yield a high load on stent members, and in particular on inner frame 305a and/or coupling arms 315a, 320a, and can even result in temporary atrial displacement of the inner frame 305a relative to the outer frame 310a and/or the native valve annulus. As should be understood, the heart beats dozens of times a minute, and a prosthetic heart valve may remain functional for years or decades. The cyclical stress put on the coupling arms 315a, 320a as the inner frame 305a is resisting atrial displacement during each heartbeat can cause early fatigue of the stent, particularly at coupling arms 315a, 320a, and thus can decrease the overall fatigue life of the valve.

It should be understood that, although not shown in FIG. 3, the inner frame 305a and/or outer frame 310a may include a skirt or cuff (e.g., tissue or fabric) along the luminal and/or abluminal surface(s) thereof that is substantially impermeable to blood flow to further seal the prosthetic heart valve 300a within the native valve annulus and to ensure that the only path for blood to flow is through the leaflets 330a when they are open.

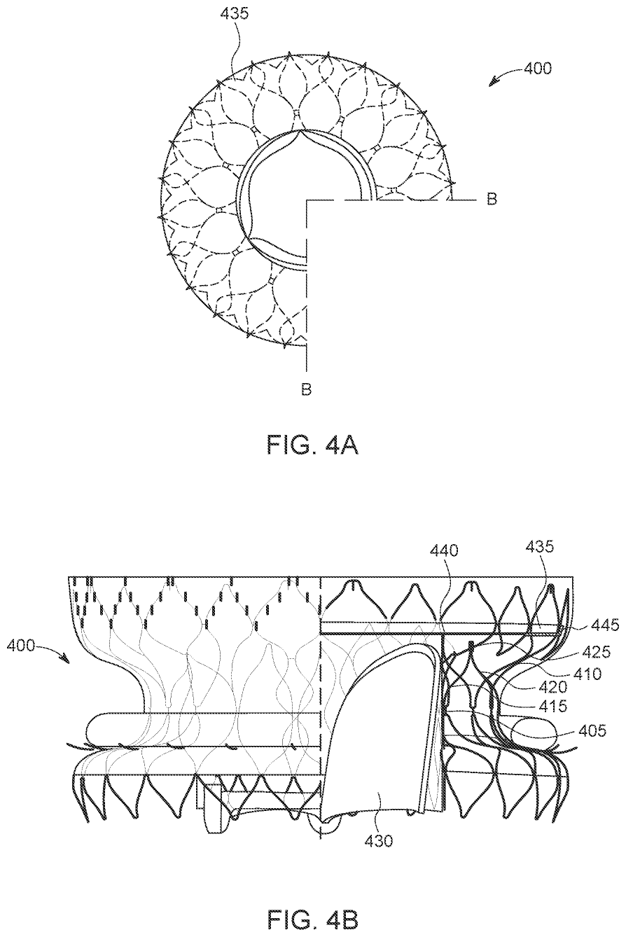

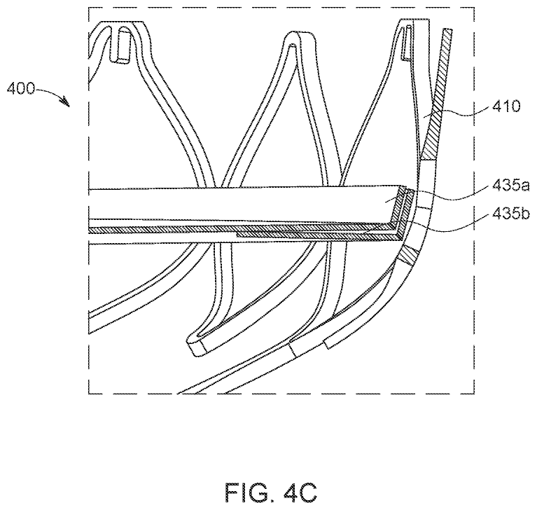

FIG. 4A is a top view of a prosthetic heart valve 400 incorporating a sealing feature 435 according to one or more aspects of the disclosure. FIG. 4B is a sectional side view along B-B of the prosthetic heart valve 400 of FIG. 4A. FIG. 4C is a sectional side view of a portion of the prosthetic heart valve 400 of FIGS. 4A-B.

As shown in FIG. 4B, the prosthetic heart valve 400 can include an inner frame 405 and an outer frame 410, similar to the inner frame and outer frame described above. The inner frame 405 can be coupled with the outer frame 410 by any type of structural engagement, for example, at least one coupling arm 415 that can be coupled to at least one respective coupling arm 420 of the outer frame 410 at an anchoring point 425. The prosthetic heart valve 400 can include one or more prosthetic leaflets 430. In the illustrated embodiment, prosthetic heart valve 400 includes three prosthetic leaflets, and the inner frame 405 includes three corresponding commissure attachment features. More or fewer prosthetic leaflets 430 may be provided, with a correspondingly larger or smaller number of commissure attachment features provided on the inner frame 405. The prosthetic leaflets 430 may be formed of any suitable material, including tissue (e.g., porcine or bovine pericardium) or fabric (e.g., synthetic fabrics such as PE, PET, PTFE, UHMWPE, etc.).

As shown in FIGS. 4B-C, the prosthetic heart valve 400 can include a sealing feature 435. As is described in greater detail below, the sealing feature 435 can include a plurality of layers 435a-b. Advantageously, the plurality of layers 435a-b can help to increase the strength of a connection (e.g. suture or sewing connection) between the sealing feature 435 and the outer frame 410.

The sealing feature 435 can couple with the inner frame 405 and the outer frame 410. For example, the sealing feature 435 can couple with the inner frame 405 at a coupling point 440 and the sealing feature 435 can couple with the outer frame 410 at a coupling point 445.

The sealing feature 435 can include a plurality of layers 435a-b, including at least a first layer 435a and a second layer 435b. The second layer 435b can be joined (e.g., sewn, sutured, ultrasonically welded, or any other attachment method) directly on a ventricular (or outflow) side of the first layer 435a. The sealing feature 435 can extend and cover at least a portion of or the entire two-dimensional annular span between the coupling point 440 at inner frame 405 and the coupling point 445 at outer frame 410.

The first layer 435a and/or second layer 435b can be formed at least in part, or in its entirety, of any suitable material that is substantially impermeable to blood and thereby prevents blood flow across the prosthetic heart valve 400 and through the sealing feature 435 when the prosthetic leaflets 430 are in the closed or coapted condition. The sealing feature 435 can be formed of a tissue or a fabric, including synthetic fabrics, such as PET, PTFE, etc. In one example, the first layer 435a and the second layer 435b are both PET fabric.

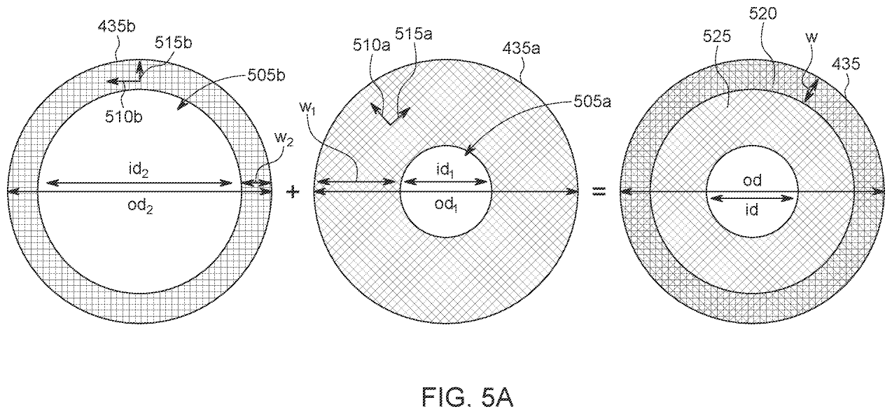

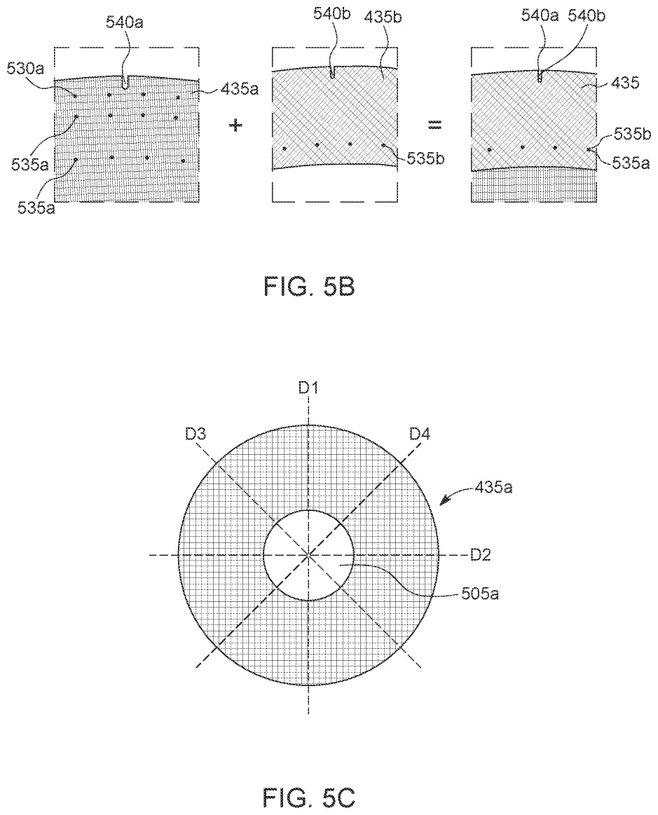

FIG. 5A is a schematic view of a plurality of layers 435a-b of sealing feature 435. As schematically shown, the first layer 435a (on the left side of the view of FIG. 5A) can be joined with the second layer 435b (in the middle of the view of FIG. 5A) to form the assembled sealing feature 435 (on the right side of the view of FIG. 5A).

As described above, the sealing feature 435 can extend and cover the entire two-dimensional annular span between the coupling point 440 at inner frame 405 and the coupling point 445 at outer frame 410. In this regard, the first layer 435a can extend and cover the entire two-dimensional annular span between the coupling point 440 at inner frame 405 and the coupling point 445 at outer frame 410, while the second layer 435b can extend and cover a portion of the two-dimensional annular span between the coupling point 440 at inner frame 405 and the coupling point 445 at outer frame 410.

The first layer 435a can define a central hole 505a having an inner diameter id₁ that approximately corresponds to an outer diameter of inner frame 405 (in the expanded condition). In one example, the diameter id₁ can be in the range of 20-30 mm.

The first layer 435a can have an outer diameter od₁ that approximately corresponds to an outer diameter of an atrial portion of outer frame 410 (in the expanded condition). In one example, the outer diameter od₁ can be in the range of 40 mm to 65 mm and in one particular example can be in the range of 48 mm to 56 mm.

The first layer 435a can have a width w₁ between the inner diameter and the outer diameter approximately corresponding to the two-dimensional annular span between the coupling point 440 at inner frame 405 and the coupling point 445 at outer frame 410. In one example, the width w₁ can be 15 mm.

The second layer 435b can define a central hole 505b having an inner diameter id₂. In this example, the inner diameter id₂ is greater than the inner diameter id₁, resulting in the central hole 505b having a larger area than the central hole 505a of the first layer 435a. In one example, the inner diameter id₂ can be approximately 46 mm.

The second layer 435b can have an outer diameter od₂ that approximately corresponds to an outer diameter of an atrial portion of outer frame 410. In this regard, the outer diameter od₁ can be equal to the outer diameter od₂, resulting in substantial alignment of the outer perimeters of the first and second layers 435a-b upon joining. In one example, the outer diameter od₂ can be in the range of 40 mm to 65 mm and in one particular example can be in the range of 48 mm to 56 mm.

The second layer 435b can have a width w₂ between the inner diameter id₂ and the outer diameter od₂ smaller than the two-dimensional annular span between the coupling point 440 at inner frame 405 and the coupling point 445 at outer frame 410. The width w₁ can be greater than the width w₂, and in one example the width w₂ can be 5 mm.

It should be understood that the diameters and widths of the layers described above are merely exemplary, and may be at least somewhat dependent on the size of the prosthetic heart valve 400. For example, if prosthetic heart valve 400 is designed for replacing a native tricuspid valve, it may have a larger outer diameter than if it were designed for replacing a native mitral valve, and thus may include relatively large sealing members 435.

After the first layer 435a is joined with the second layer 435b, the sealing feature 435 has an overlap region 520 extending inwardly from an outer perimeter of the sealing feature 435, in which the first layer 435a and the second layer 435b overlap. The sealing feature 435 also has a region 525, positioned radially inwardly of the overlap region 520 and extending to the central hole 505a, where the first layer 435a and the second layer 435b do not overlap. The overlap region 520 can have a width w equivalent to the width w₂ of the second layer 435b. The sealing feature 435 can have an inner diameter id that is equal to the inner diameter id₁ and can have an outer diameter od that is equal to both outer diameters od₁ and od₂.

In the illustrated example, the first layer 435a and the second layer 435b are each formed as a woven fabric, preferably with a tight enough weave to prevent blood from flowing across either fabric. The first layer 435a can have a first weave pattern defined by a first warp defining a first warp direction 510a and a first weft defining a first weft direction 515a, and the second layer 435b can have a second weave pattern defined by a second warp defining a second warp direction 510b and a second weft defining a second weft direction 515b. In general, the first warp direction 510a is perpendicular (e.g., 90 degrees) to the first weft direction 515a and the second warp direction 510b is perpendicular (e.g., 90 degrees) to the second weft direction 515b.

In one example, after the first layer 435a is joined, coupled, or otherwise assembled to the second layer 435b, the first weave pattern of the first layer 435a is offset from the second weave pattern of the second layer 435b. In one particular example, the offset represents an angular offset between the first warp direction 510a of the first layer 435a and the second warp direction 510b of the second layer 435b, with a corresponding offset between the first weft direction 515a of the first layer 435a and the second weft direction 515b of the second layer 435b. In one particular example, the angular offset is approximately 45 degrees.

In general, where a load is applied to the first layer 435a or the second layer 435b along the weave (e.g., along the warp direction or the weft direction), the fabric is the stiffest and exhibits the lowest stretchiness. Where the load is applied at 45 degrees to the weave (e.g., 45 degrees relative to the warp direction or the weft direction), the fabric is least stiff and exhibits the highest stretchiness. It should be understood that the threads or filaments that form the warp and weft of the sealing feature 435 may not be elastic and thus may not be capable of stretching to any significant degree. When a woven fabric has an annular shape, with an inner perimeter attached to a structure and an outer perimeter attached to another structure, some of the filaments or threads of the weave will extend in a straight line along a diameter of the annular shape. For example, referring to FIG. 5C, warp fibers extend along diameter direction D1 and weft fibers extend along direction D2. If an upward force (e.g. in a direction into or out of the page in the view of FIG. 5C) is applied to the sealing feature 435, that force may tend to move the structure within the hole 505a upwards relative to the structure coupled to the outer perimeter of the first layer. This may occur, for example, if the outer perimeter of the sealing feature 435 is coupled to the outer frame 410 which is secured to a native valve annulus, and the inner perimeter of the sealing feature 435 is coupled to the inner frame 405 which is more prone to movement. For example, referring again briefly to FIG. 4B, during ventricular systole, the prosthetic leaflets 430 will close and ventricular pressure will tend to push the inner frame 405 upward relative to the outer frame 410. Referring back to FIG. 5C, when this force is applied, the warp fibers extending along diameter direction D1 and weft fibers extending along diameter direction D2 sustain a maximum force. This is because, at least in part, the weave pattern is incapable of stretching (to any meaningful degree) along these directions. However, the opposite is true for the weave along diameter directions D3 and D4, which directions are oriented at about 45 degrees to both the warp and weft directions. In other words, the weave pattern along diameter directions D3 and D4 is capable of maximum stretching, and thus these areas of the weave pattern will sustain a minimum force. It has been determined that the difference between the maximum stress and the minimum stress may be a 10-20 fold difference, including about a 15-fold difference. Over time, the large stress on the first layer 435a along diameter directions D1 and D2, compared to the relatively small stress along diameter directions D3 and D4, may lead to failure of the first layer 435a in the areas of high stress.

In order to reduce the highly uneven application of stress to the different areas of the sealing feature 435, the second layer 435b may be assembled to the first layer 435a at the above-described 45-degree offset. Advantageously, implementation of the angular offset between the first weave direction and the second weave direction allows for more even distribution of load circumferentially around the sealing feature 435, while increasing (e.g., doubling) a thickness of the sealing feature in the overlap region 520, which can experience higher stress, as compared to the region 525. This increased thickness may help to provide additional reinforcement. With the above-described implementation, areas of the first layer 435a that would experience maximum stress are aligned with areas of the second layer 435b that would experience minimum stress, and vice versa. When the first and second layers 435a, 435b are assembled in this fashion, there is less variation in local stress applied to the assembled sealing feature 435, making it less likely that any one particular area will be prone to premature failure from long-term exposure to high stresses.

FIG. 5B is a schematic view of first and second layers 435a, 435b and sealing feature 435. As shown, the first layer 435a has a plurality of circumferentially oriented suture holes 530a and 535a. The suture holes 530a are positioned radially outwardly relative to the suture holes 535a and are positioned to couple the sealing feature 435 to the outer frame 410 (e.g., via a skirt attached to the outer frame 410). The suture holes 535a are positioned to join the first and second layers 435a, 435b directly to one another. The first layer 435a also has a notch 540a at an outer perimeter thereof.

The second layer 435b has a plurality of suture holes 535b and a notch 540b at an outer perimeter thereof. During joining of the first layer 435a and the second layer 435b, the notches 540a and 540b provide a visual indicator of alignment between the layers 435a, 435b. In this regard, alignment of the notches 540a, 540b also provides for alignment of the suture holes 535a and 535b and also aligns the layers in the predetermined angular offset (e.g. 45 degrees) of the first weave pattern and the second weave pattern. The sealing feature 435 can thus be joined through suture holes 535a, 535b that are aligned. In other examples, the first layer 435a and second layer 435b can be joined by any attachment method, such as ultrasonic welding, or a combination of attachment methods.

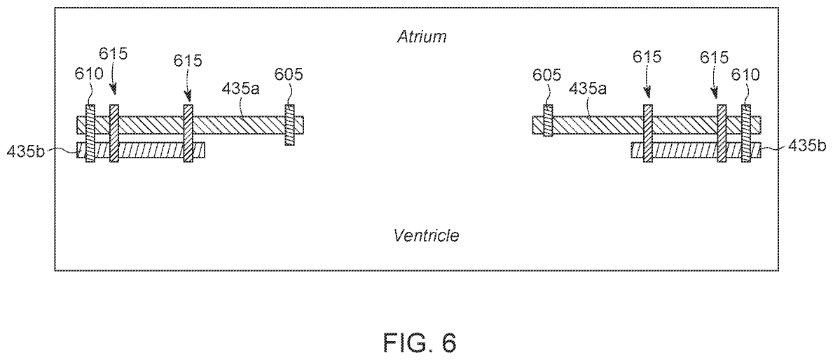

FIG. 6 is a schematic cross-sectional diagram of the first layer 435a and the second layer 435b. As shown, the first layer 435a can couple to the inner frame 405 via a coupling 605, which can be a single running stitch pattern. The first layer 435a and the second layer 435b can both be sutured to the outer frame 410 via coupling 610, which can be a single running stitch pattern. The first layer 435a and the second layer 435b can be directly coupled to one another via a coupling 615, which can include a double running stitch pattern, with one suture positioned radially outwardly relative to the other.

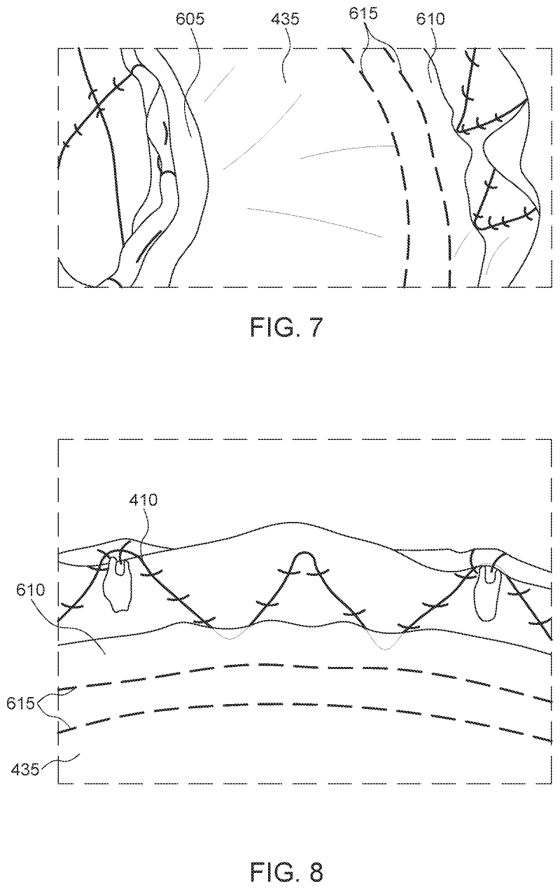

FIGS. 7 and 8 are perspective views of the sealing feature 435 showing the coupling 615. As shown, the coupling 615 can include a double running stitch pattern. The sealing feature 435, including both first layer 435a and second layer 435b, can be coupled to the outer frame 410 at coupling 610 (e.g., via a skirt), which can be a single running stitch pattern. A portion of the sealing feature 435 extending radially outward from the outermost suture holes 535a can be folded in an atrial direction to allow suture holes 530a to align with the skirt associated with the outer frame 410.

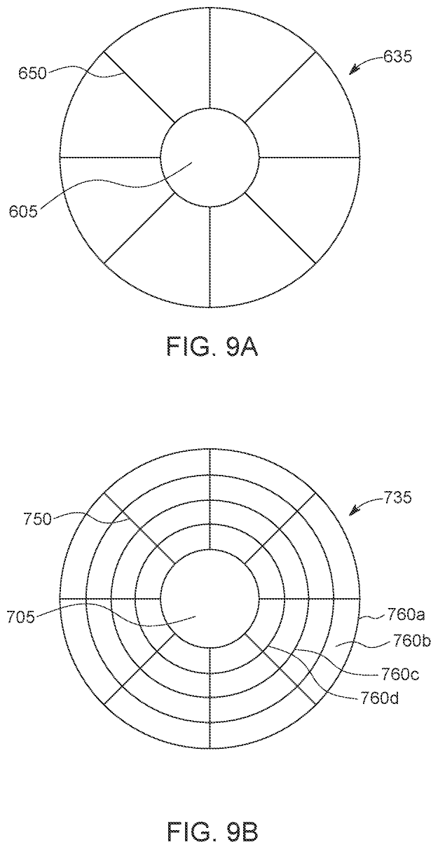

Although one way to better distribute the force encountered via a sealing feature is to angularly offset weave patterns of two separate layers, still other configurations may help reduce variations in stress along different portions of a sealing fabric. For example, FIG. 9A illustrates an alternate sealing member 635 that has a plurality of filaments or threads in the form of spokes 650 all extending along a diameter of the sealing member 635, between the inner perimeter that defines the central hole 605 to the outer perimeter. In practice, sealing member 635 would need to include enough spokes 650 (or a separate sealing layer) to ensure that blood cannot pass through the sealing member 635. As should be understood, since each spoke 650 extends along a diameter direction, there would be little variation in stresses encountered by the individual spokes 650. FIG. 9B illustrates a similar embodiment, where sealing member 735 includes spokes 750 extending from the inner perimeter defining central hole 705 to the outer perimeter. However, sealing member 735 includes additional filaments or threads 760a-d extending concentrically to the outer and inner perimeters of the sealing member 735 in a general spider-web configuration. These additional filaments or threads 760a-d may provide for additional reinforcement, while still maintaining low variability in stress encountered by any one (or any one group) of filaments or threads of the sealing member 735. While FIG. 9B depicts threads 760a-d being spaced from one another in a spider web configuration, in another example, a single thread is configured in a continuous spiral configuration relative to the spokes 750.

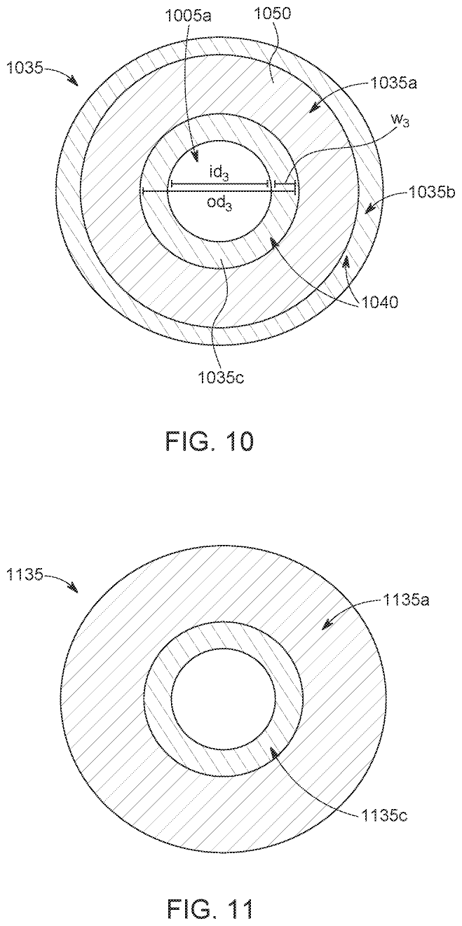

FIG. 10 illustrates an exemplary sealing feature 1035 according to one or more aspects of the disclosure. In this example, the sealing feature 1035 includes a first layer 1035a that can be identical to or similar to layer 435a described above and a second layer 1035b that can be identical to or similar to second layer 435b described above. In addition, the sealing feature 1035 can include a third layer 1035c.

The third layer 1035c can define a central hole 1005a having an inner diameter id₃ that approximately corresponds to an outer diameter of the inner frame 405 (in the expanded condition). In one example, the inner diameter id₃ can be the same as the inner diameter of the first layer 1035a. In one example, the diameter id₃ can be in the range of 20-30 mm.

The third layer 1035c can have an outer diameter od₃ that that is less than the outer diameter of first layer 1035a and less than both the inner and outer diameters of second layer 1035b. The third layer 1035c can have a width w₃ between the inner diameter id₃ and the outer diameter od₃. The width w₃ can be greater than, less than, or equal to a width of the second layer 1035b. In this configuration, the sealing feature 1035 defines two regions 1040 comprising a plurality of layers with a gap region 1050 therebetween comprising a single layer (e.g., first layer 1035a).

The third layer 1035c can be joined to first layer 1035a according to any attachment method, e.g., sewn, sutured, ultrasonically welded, or any other attachment method. The third layer 1035c can be formed at least in part, or in its entirety, of any suitable material that is substantially impermeable to blood and thereby prevents blood flow across the prosthetic heart valve and through the sealing feature 1035 when the prosthetic leaflets are in the closed or coapted condition. The sealing feature 1035 can be formed of a tissue or a fabric, including synthetic fabrics, such as PET, PTFE, etc. In one example, the first layer 1035a, the second layer 1035b, and the third layer 1035c are each PET fabric. The third layer 1035c can be formed as a woven fabric, preferably with a tight enough weave to prevent blood from flowing across layers 1035a-c. As in the examples above, the first layer 1035a can have a first weave pattern defined by a first warp defining a first warp direction and a first weft defining a first weft direction, the second layer 1035b can have a second weave pattern defined by a second warp defining a second warp direction and a second weft defining a second weft direction, and the third layer 1035b can have a third weave pattern defined by a third warp defining a third warp direction and a third weft defining a third weft direction. In this regard, the first weave pattern is offset from both the second weave pattern and the third weave pattern, with the second weave pattern and the third weave pattern being aligned or substantially aligned with each other.

Advantageously, the regions 1040 can provide additional reinforcement in the sealing feature 1035, making it less likely that any one particular area will be prone to premature failure from long-term exposure to high stresses, while also reducing overall material and therefore having reduced packing volume and/or sheathing forces.

Further advantageously, the regions 1040 provide redundancy in the event of a breakage in the sealing feature 1035. In this regard, if one of the regions 1040 experiences breakage or tearing, is it effectively replaced by a region having increased stretchiness that is more suitable for loading the valve into a delivery member.

FIG. 11 illustrates another exemplary sealing feature 1135 according to one or more aspects of the disclosure. In this example, the sealing feature 1135 includes a first layer 1135a similar to layers 435a, 1035a described above and a third layer 1035c similar to layer 1035c described above, but omits a second layer (e.g., second layer 1035b).

Advantageously, the region of third layer 1135c can provide additional reinforcement in the sealing feature 1135, making it less likely that any one particular area will be prone to premature failure from long-term exposure to high stresses, while also reducing overall material and therefore having reduced packing volume and/or sheathing forces.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
an inner frame having a longitudinal axis;
a plurality of prosthetic leaflets coupled to the inner frame, the plurality of prosthetic leaflets configured to allow blood to flow through the inner frame in an antegrade direction along the longitudinal axis and to substantially block blood from flowing through the inner frame in a retrograde direction along the longitudinal axis;
an outer frame connected to the inner frame; and
a sealing feature coupled to the inner frame at a coupling point and extending between the coupling point and a point of the outer frame that is radially outward of the coupling point in a direction substantially orthogonal to the longitudinal axis, the sealing feature comprising a plurality of layers.

2. The prosthetic heart valve of claim 1, wherein the sealing feature comprises a first layer and a second layer, the first layer and the second layer each being formed as woven layers.

3. The prosthetic heart valve of claim 2, wherein there is an offset between a first weave pattern of the first layer and a second weave pattern of the second layer.

4. The prosthetic heart valve of claim 3, wherein the offset comprises an angular offset between the first weave pattern and the second weave pattern.

5. The prosthetic heart valve of claim 4, wherein the first layer defines a first notch and the second layer defines a second notch, such that alignment of the first notch and the second notch results in the angular offset.

6. The prosthetic heart valve of claim 4, wherein the first weave pattern includes a first warp and first weft oriented at 90 degrees relative to each other, the second weave pattern includes a second warp and second weft oriented at 90 degrees relative to each other, and the angular offset is 45 degrees.

7. The prosthetic heart valve of claim 6, wherein the first layer and the second layer are each substantially impermeable to blood flowing through the respective layer.

8. The prosthetic heart valve of claim 7, wherein when the prosthetic heart valve is in an implanted condition during ventricular systole, areas of the first layer experiencing a maximum stress are aligned with areas of the second layer experiencing a minimum stress.

9. The prosthetic heart valve of claim 1, wherein the sealing feature comprises an overlap region at which a first layer of the plurality of layers overlaps a second layer of the plurality of layers.

10. The prosthetic heart valve of claim 9, wherein the sealing feature comprises a second region at which the first layer and the second layer do not overlap.

11. The prosthetic heart valve of claim 10, wherein the second region is positioned radially inwardly relative to the overlap region.

12. The prosthetic heart valve of claim 10, wherein the overlap region has a greater thickness than the second region.

13. The prosthetic heart valve of claim 1, wherein a first layer of the plurality of layers and a second layer of the plurality of layers are coupled to the outer frame.

14. The prosthetic heart valve of claim 1, wherein a first layer of the plurality of layers and a second layer of the plurality of layers are coupled directly to each other.

15. The prosthetic heart valve of claim 14, wherein the first layer and the second layer are coupled directly to each other by a double running stitch pattern.

16. The prosthetic heart valve of claim 1, wherein a second layer of the plurality of layers has an inner diameter that is greater than an inner diameter of a first layer of the plurality of layers.

17. The prosthetic heart valve of claim 16, wherein the second layer is positioned nearer an inflow end of the prosthetic heart valve than is the first layer.
